(19) United States
(12) Patent Application Publication
(10) Pub. No.: US 2026/0166209 A1
(43) Pub. Date: Jun. 18, 2026

(54) BLOOD PURIFICATION SYSTEM AND INTERMEDIATE SYSTEM

(71) Applicant: JMS CO., LTD., Hiroshima (JP)

(72) Inventor: Kazuma SUNAMI, Hiroshima (JP)

(21) Appl. No.: 19/125,118

(22) PCT Filed: Oct. 26, 2023
(86) PCT No.: PCT/JP2023/038645

§ 371 Date: Apr. 28, 2025

(30) Foreign Application Priority Data
Oct. 28, 2022 (JP) 2022-173786

(51) Int. Cl. A61M 1/16; A61M 1/36

(52) U.S. Cl. CPC A61M 1/1698; A61M 1/3627; A61M 1/3666

(57) ABSTRACT

The blood purification system connected to the ECMO system having an ECMO blood pump and an artificial lung includes a water removal control unit that maintains the concentration of blood circulating through the blood purification system at a predetermined concentration when the rate of change of the flow of blood in the ECMO system exceeds a predetermined threshold.

### TECHNICAL FIELD

The present invention relates to a blood purification system that is connected to an extracorporeal membrane oxygenation system, and relates to an intermediate system that is adapted to be provided at a connection between an extracorporeal membrane oxygenation system and a blood purification system.

### BACKGROUND ART

In the area of intensive care, treatment using an extracorporeal membrane oxygenator (hereinafter, also referred to as ECMO) is performed on a patient with weakened heart or lung function. Some such patients require continuous renal replacement therapy (hereinafter also referred to as CRRT) for blood purification due to complications of renal damage. A patient undergoing the ECMO treatment is administered with an anticoagulant within the ECMO circuit and is prone to bleeding. When blood is drained from and returned to a different blood vessel of the patient for CRRT, it may be necessary to administer an anticoagulant also in the CRRT circuit, which is not preferable because the bleeding tendency increases. Furthermore, in many cases, a plurality of catheters are inserted into a central vein for the purpose of systemic management, in addition to the drainage and return cannulas for ECMO, and it is difficult to newly insert a vascular access for CRRT. Therefore, attempts to connect a CRRT circuit directly to an ECMO circuit have been made at the clinical site (see Patent Document 1).

### CITATION LIST

### Patent Document

Patent Document 1: Japanese Unexamined Patent Application (Translation of PCT Application), Publication No. 2010-528781

### DISCLOSURE OF THE INVENTION

### Problems to be Solved by the Invention

In a state where a CRRT system is connected to an ECMO system, when a problem occurring in the ECMO system makes the circulation unstable, the CRRT system is affected by the ECMO system and stops the circulation therein. For this reason, a thrombus is likely to form in the CRRT circuit. If the operation of the CRRT system is restarted after the recovery of the ECMO system, the thrombus that has formed in the CRRT circuit may be transferred to the ECMO circuit. The thrombus may cause a failure of the circulation in the CRRT circuit.

In view of the circumstances described above, an object of the present invention is to provide a blood purification system and an intermediate system that are adapted for an application in which an ECMO system is connected to the blood purification system, and are capable of maintaining circulation in the blood purification system during recovery of the ECMO system after a problem has occurred in the ECMO system.

### Means for Solving the Problems

The present invention relates to a blood purification system that is connected to an extracorporeal membrane oxygenation system including an ECMO blood pump and an oxygenator disposed downstream of the ECMO blood pump. The blood purification system includes: a blood drainage line having an upstream end that is connected to the extracorporeal membrane oxygenation system; a blood purifier connected to a downstream end of the blood drainage line; a blood return line having an upstream end connected to the blood purifier and a downstream end that is connected to the extracorporeal membrane oxygenation system; a flowmeter configured to measure a flow rate in the extracorporeal membrane oxygenation system; and a controller. The controller includes a moisture removal controller that maintains a concentration of liquid flowing through the blood purification system at a predetermined concentration in a case where a rate of change of the flow rate measured by the flowmeter exceeds a predetermined threshold.

Preferably, the blood purification further includes: a flow rate adjuster provided to the blood drainage line and configured to adjust a flow rate in the blood drainage line; and a pressure gauge configured to measure a pressure at a connection between the extracorporeal membrane oxygenation system and the blood drainage line or the blood return line, and the controller further includes a circulation controller that reduces a flow rate of the flow rate adjuster to a predetermined flow rate in a case where a rate of change of the flow rate measured by the flowmeter exceeds the predetermined threshold and the pressure measured by the pressure gauge is within a predetermined range.

Preferably, the blood purification system further includes: a bypass line connecting a point upstream of the blood purification pump in the blood drainage line to the blood return line; a first flow path switch disposed in a vicinity of a connection between the blood drainage line and the bypass line; and a second flow path switch disposed in a vicinity of a connection between the blood return line and the bypass line, and the controller includes a switch controller. In a case where a rate of change of the flow rate measured by the flowmeter exceeds the predetermined threshold and the pressure measured by the pressure gauge exceeds the predetermined range, the switch controller switches the second flow path switch so as to switch a flow path for liquid flowing through the blood return line to the bypass line, and switches the first flow path switch to stop an inflow of liquid from the extracorporeal membrane oxygenation system to the blood drainage line and to causes the liquid flowing through the bypass line to flow into the blood drainage line.

Preferably, the blood purification system further includes a blood return pump provided to the blood return line, the upstream end of the blood drainage line is connected to a point downstream of the ECMO blood pump in the extracorporeal membrane oxygenation system, and the downstream end of the blood return line is connected to a point downstream of the ECMO blood pump in the extracorporeal membrane oxygenation system.

Preferably, the blood purification system further includes: a pressure buffer provided at a point upstream of the blood return pump in the blood return line and capable of storing a predetermined amount of liquid; and a buffer pressure gauge configured to measure a pressure of the pressure buffer, and the controller further includes a flow rate controller that adjusts a flow rate of the flow rate adjuster and a flow rate of the blood return pump so as to control a value measured by the buffer pressure gauge to be within a predetermined range.

The present invention also relates to an intermediate system adapted to be provided at a connection between an extracorporeal membrane oxygenation system that includes an ECMO blood pump and an oxygenator disposed downstream of the ECMO blood pump, and a blood purification system that includes a blood purification pump, a blood purifier disposed downstream of the blood purification pump, a drain line for draining filtrate from the blood purifier, and a drain pump provided to the drain line. The intermediate system includes: an intermediate blood drainage line having an upstream end that is connected to a point downstream of the ECMO blood pump in the extracorporeal membrane oxygenation system, and a downstream end that is connected to an upstream end of the blood purification system; a flow rate adjuster provided to the intermediate blood drainage line and configured to adjust a flow rate in the intermediate blood drainage line; an intermediate blood return line having an upstream end that is connected to a downstream end of the blood purification system, and a downstream end that is connected to a point downstream of the ECMO blood pump in the extracorporeal membrane oxygenation system; a blood return pump provided to the intermediate blood return line; a flowmeter configured to measure a flow rate in the extracorporeal membrane oxygenation system; a notifier; and a controller. In a case where a rate of change of the flow rate measured by the flowmeter exceeds a predetermined threshold, the controller determines that a problem has occurred in the extracorporeal membrane oxygenation system, and causes the notifier to notify the occurrence of the problem.

Preferably, the intermediate system further includes: a bypass line connecting a point upstream of the flow rate adjuster in the intermediate blood drainage line to the blood return line; a first flow path switch disposed in a vicinity of a connection between the intermediate blood drainage line and the bypass line; a second flow path switch disposed in a vicinity of a connection between the intermediate blood return line and the bypass line; and a pressure gauge configured to measure a pressure at a connection between the extracorporeal membrane oxygenation system and the intermediate blood drainage line or the intermediate blood return line. The controller preferably includes a switch controller, and in a case where a rate of change of the flow rate measured by the flowmeter exceeds the predetermined threshold and the pressure measured by the pressure gauge exceeds a predetermined range, the switch controller switches the second flow path switch so as to switch a flow path for liquid flowing through the intermediate blood return line to the bypass line, and switches the first flow path switch to stop an inflow of liquid from the extracorporeal membrane oxygenation system to the intermediate blood drainage line and to causes the liquid flowing through the bypass line to flow into the intermediate blood drainage line.

### Effects of the Invention

The present invention can provide a blood purification system and an intermediate system that are capable of maintaining circulation in the blood purification system by means of a circulation controller and a moisture removal flow rate controller in a case where a problem occurs in an ECMO system connected to the blood purification system.

### BRIEF DESCRIPTION OF THE DRAWINGS

FIG. 1 is a diagram schematically illustrating a configuration of an ECMO system and that of a CRRT system according to a first embodiment of the present invention;

FIG. 2 is a block diagram of the CRRT system according to the first embodiment of the present invention;

FIG. 3 is a diagram illustrating an operation state of the CRRT system in a case where a minor problem has occurred in the ECMO system according to the first embodiment of the present invention;

FIG. 4 is a diagram illustrating an operation state of the CRRT system in a case where a severe problem has occurred in the ECMO system according to the first embodiment of the present invention;

FIG. 5 is a diagram schematically illustrating a configuration of an ECMO system and that of a CRRT system according to a second embodiment of the present invention;

FIG. 6 is a block diagram of the CRRT system according to the second embodiment of the present invention;

FIG. 7 is a diagram illustrating an operation state of the CRRT system in a case where a minor problem has occurred in the ECMO system according to the second embodiment of the present invention;

FIG. 8 is a diagram illustrating an operation state of the CRRT system in a case where a severe problem has occurred in the ECMO system according to the second embodiment of the present invention;

FIG. 9 is a diagram schematically illustrating a configuration of an ECMO system that of a CRRT system, and that of an intermediate system according to a third embodiment of the present invention;

FIG. 10 is a block diagram of the CRRT system and the intermediate system according to the third embodiment of the present invention;

FIG. 11 is a diagram illustrating an operation state of the CRRT system and the intermediate system in a case where a minor problem has occurred in the ECMO system according to the third embodiment of the present invention; and

FIG. 12 is a diagram illustrating an operation state of the CRRT system and the intermediate system in a case where a severe problem has occurred in the ECMO system according to the third embodiment of the present invention.

### PREFERRED MODE FOR CARRYING OUT THE INVENTION

Preferred embodiments of a continuous renal replacement therapy system as a blood purification system of the present invention and an intermediate system will be described with reference to the drawings. The intermediate system of the present invention is provided at a connection between an extracorporeal membrane oxygenation (ECMO) system and a continuous renal replacement therapy (CRRT) system. The ECMO system described herein is applied to a patient with weakened heart or lung function, pumps the blood out from the patient, sends the blood to an oxygenator, and returns the blood oxygenated by the oxygenator to the patient, thereby assisting the patient's heart or lung function. The CRRT system is applied to a patient with acute renal dysfunction, a patient with sepsis or plethora, and a patient with a similar disease, and removes waste products and moisture from the blood little by little over time so as not to abruptly change the blood concentration, blood circulation volume, and blood pressure. The CRRT includes continuous hemodialysis (CHD), continuous hemofiltration (CHF), and continuous hemodiafiltration (CHDF). In the embodiments described below, a case of employing the continuous hemodiafiltration (CHDF) will be described as an example. In the present embodiment, the blood purification system of the present invention is applied to a CRRT system.

However, the present invention is not limited thereto. For example, the blood purification system may be applied to a direct hemoperfusion (DHP) system. The direct hemoperfusion is a therapy in which an inflammatory substance is adsorbed and removed by an adsorption type blood purifier functioning as a blood purifier. Alternatively, the blood purification system may be applied to a plasma adsorption therapy system.

### First Embodiment

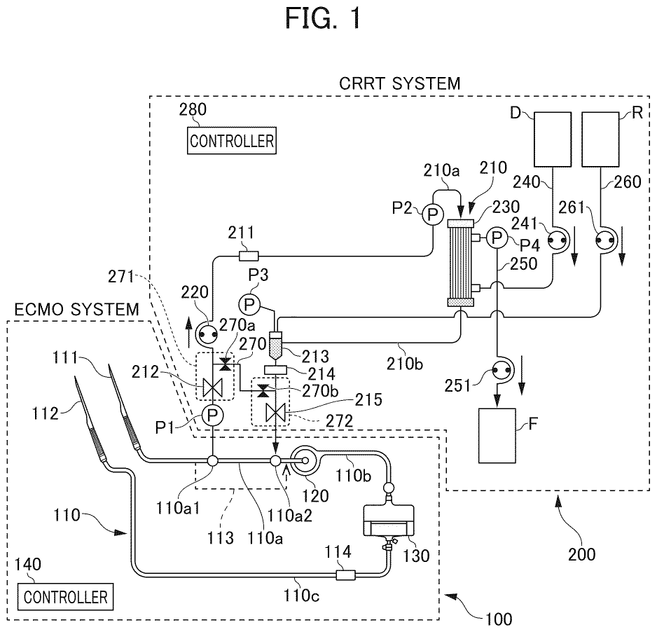

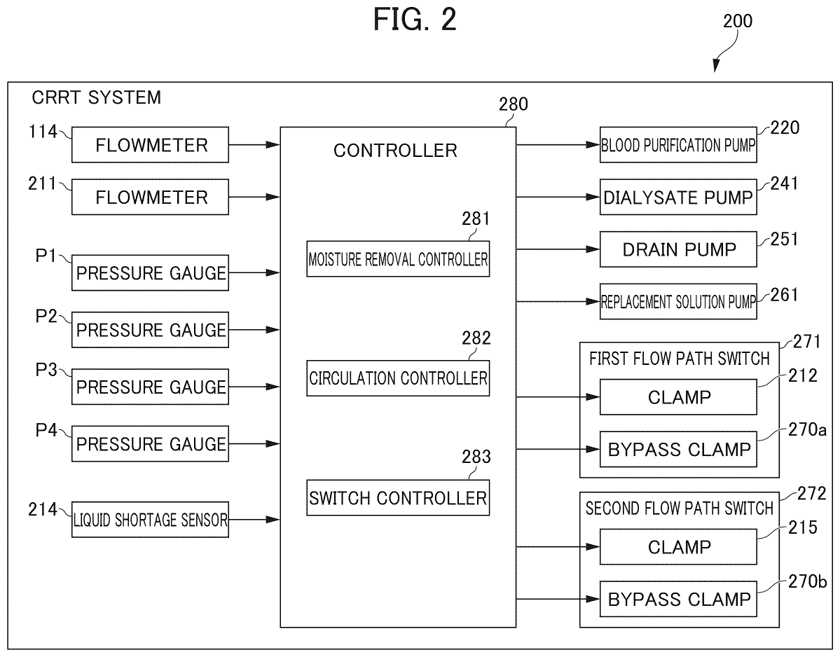

A first embodiment will be described in detail with reference to FIGS. 1 to 4. FIG. 1 is a diagram schematically illustrating a configuration of an ECMO system 100 and that of a CRRT system 200 according to the first embodiment of the present invention. FIG. 2 is a block diagram of the CRRT system 200.

### EMO System

As illustrated in FIG. 1, the ECMO system 100 includes an ECMO blood circuit 110, an ECMO blood pump 120, an oxygenator 130, and a controller 140. The ECMO blood circuit 110 is a circuit for extracorporeally circulating the blood of a patient, and includes a blood drainage line 110a, a connection line 110b, and a blood return line 110c. The blood drainage line 110a has one end connected to a blood drainage cannula 111 and the other end connected to the ECMO blood pump 120. The blood drainage line 110a has a first branch portion 110a1 and a second branch portion 110a2. A connector such as a three-way stopcock is attached to each of the first branch portion 110a1 and the second branch portion 110a2. The connection line 110b has one end connected to the ECMO blood pump 120 and the other end connected to the oxygenator 130. In the present embodiment, the blood drainage line 110a is provided with a bypass line 113 that bypasses the first branch portion 110a1 and the second branch portion 110a2. The connection between the bypass line 113 and the blood drainage line 110a is constituted by, for example, a Y-connector (not shown).

Provision of the bypass line 113 allows the ECMO system to operate more safely by switching to the bypass line 113 from the flow path from the blood drainage cannula 111 to the ECMO blood pump 120 in a case where the operation of the CRRT system 200 is stopped due to some malfunction occurring therein. The blood return line 110c has one end connected to the oxygenator 130 and the other end connected to a blood return cannula 112. A flowmeter 114 for monitoring an operation state of the ECMO system 100 is attached to the blood return line 110c. In the present embodiment, an ultrasonic flowmeter is used as the flowmeter 114. An optical flowmeter or the like may be used as the flowmeter 114.

The ECMO blood pump 120 draws the blood from the patient's vein via the blood drainage cannula 111 and the blood drainage line 110a. The drawn blood is sent to the oxygenator 130 through the connection line 110b, and thereafter, passes through the blood return line 110c to be returned to the patient via the blood return cannula 112. A known centrifugal pump or roller pump is used as the ECMO blood pump 120.

The oxygenator 130 includes a hollow fiber membrane (not shown) formed by bundling hollow fibers having a large number of micropores, and oxygenates the blood pumped from the ECMO blood pump 120 and removes carbon dioxide by causing oxygen to flow inside the hollow fibers and causing the blood to flow outside the hollow fibers. Furthermore, the oxygenator 130 traps air bubbles present in the blood to be returned and prevents the air bubbles from flowing to a downstream portion of the ECMO circuit. As the oxygenator 130, a known membrane oxygenator is used. The oxygenator 130 may have a heat exchange function.

The controller 140 is constituted by an information processing apparatus (computer), and controls the operation of the ECMO system 100 by driving each pump included in the ECMO system 100 by executing a control program.

### CRRT System

As illustrated in FIGS. 1 and 2, the CRRT system 200 includes a CRRT blood circuit 210, a blood purification pump 220, a blood purifier 230, a dialysate supply line 240, a dialysate drain line 250, a replacement solution line 260, a bypass line 270, and a controller 280.

The CRRT blood circuit 210 is a circuit for circulating the drawn blood and includes a blood drainage line 210a and a blood return line 210b. The blood drainage line 210a has one end (upstream end) connected to the first branch portion 110a1 provided to the blood drainage line 110a of the ECMO system 100, and the other end (downstream end) connected to the blood purifier 230. Although the upstream end of the blood drainage line 210a may be connected to any position of the ECMO blood circuit 110, it is connected to a point upstream of the ECMO blood pump 120 as an example in the present embodiment. The blood drainage line 210a is provided with the blood purification pump 220 as a flow rate adjuster. A pressure gauge P1 is attached to a point upstream of the blood purification pump 220, and a pressure gauge P2 is attached to a point downstream of the blood purification pump 220. Since the pressure gauge P1 reflects a pressure at the connection between the blood drainage line 210a and the ECMO blood circuit 110, it can be used to monitor a circuit internal pressure of the ECMO blood circuit 110. A flowmeter 211 is attached to the blood drainage line 210a to monitor whether the blood is being sent toward the blood purifier 230 at a normal flow rate. In the present embodiment, an ultrasonic flowmeter is used as the flowmeter 211. An optical flowmeter or the like may be used as the flowmeter 211. A clamp 212 for interrupting an inflow of the blood from the ECMO blood circuit is attached to the blood drainage line 210a between the blood purification pump 220 and the pressure gauge P1.

The blood return line 210b has one end (upstream end) connected to the blood purifier 230, and the other end (downstream end) connected to the second branch portion 110a2 provided to the blood drainage line 110a of the ECMO system 100. Here, in order to operate the CRRT system 200 without being affected by a high positive pressure, the blood return line 210b needs to be connected to a point upstream of the ECMO blood pump 120 which is a negative pressure portion in the ECMO blood circuit 110. The blood return line 210b has a drip chamber 213, a liquid shortage sensor 214, and a clamp 215 that are attached in this order from the upstream side, and the drip chamber 213 has a pressure gauge P3 attached thereto. The drip chamber 213 stores therein a certain amount of the blood in order to remove air bubbles mixed in the blood, coagulated blood, and the like. The pressure gauge P3 measures a circuit internal pressure of the blood return line 210b. Since the pressure gauge P3 reflects a pressure at the connection between the blood return line 210b and the ECMO blood circuit 110, it can be used to monitor the circuit internal pressure of the ECMO blood circuit 110. In the present embodiment, the pressure gauge P3 is attached to the drip chamber 213 as an example, but the present invention is not limited thereto. For example, the pressure gauge P3 may be attached to a point downstream of the drip chamber 213 in the blood return line 210b to measure the circuit internal pressure.

The blood purification pump 220 draws the blood from the ECMO system 100 and adjusts the flow rate of the blood flowing through the blood drainage line 210a. The drawn blood is sent to the blood purifier 230 through the blood drainage line 210a, and thereafter, passes through the blood return line 210b to be returned to the ECMO system 100.

The blood purifier 230 includes a dialysis membrane (not shown) accommodated in a cylindrical container body. The interior of the container body is partitioned into a blood-side flow path and a dialysate-side flow path (both are not shown) by the dialysis membrane, and the blood is purified by way of transfer of moisture and waste products from the blood-side flow path to the dialysate-side flow path via the dialysis membrane.

The dialysate supply line 240 is provided with a dialysate pump 241, supplies a dialysate to the blood purifier 230, and connects a dialysate source D to the dialysate-side flow path of the blood purifier 230. As the dialysate pump 241, a known roller pump or finger pump is used.

The dialysate drain line 250 is provided with a drain pump 251 and drains the dialysate out of the blood purifier 230.

The dialysate drain line 250 connects the dialysate-side flow path of the blood purifier 230 to a drainage reservoir F. As the drain pump 251, a known roller pump or finger pump is used. A pressure gauge P4 is attached to the dialysate drain line 250.

The replacement solution line 260 is provided with a replacement solution pump 261 and supplies a replacement solution from a replacement solution source R to the blood return line 210b via the drip chamber 213 provided to the blood return line 210b. As the replacement solution, a dialysate or physiological saline is used. The replacement solution may be supplied to the blood drainage line 210a between the blood purification pump 220 and the blood purifier 230.

The bypass line 270 connects the blood drainage line 210a to the blood return line 210b and is used to disconnect the CRRT system 200 from the ECMO system 100. The bypass line 270 is connected to a point upstream of the blood purification pump 220 in the blood drainage line 210a and is connected to a downstream point in the blood return line 210b. The bypass line 270 has a bypass clamp 270a in the vicinity of the connection with the blood drainage line 210a and a bypass clamp 270b in the vicinity of the connection with the blood return line 210b. The bypass clamp 270a and the clamp 212 provided to the blood drainage line 210a together constitute a first flow path switch 271. The bypass clamp 270b and the clamp 215 provided to the blood return line 210b together constitute a second flow path switch 272. Although each of the first flow path switch 271 and the second flow path switch 272 of the present embodiment is constituted by the two clamps, each of the switches may be constituted by a three-way stopcock or the like.

The controller 280 is constituted by an information processing apparatus (computer) and executes a control program to drive the pumps included in the CRRT system 200 to control the blood flow rate, the dialysate amount, and the drainage amount for the blood purifier 230, thereby performing continuous hemodiafiltration (CHDF). The controller 280 may control and drive the pumps based on flow rates measured by a metering unit (not shown) that monitors a flow rate of each of the dialysate pump 241 disposed in the dialysate supply line 240, the drain pump 251 disposed in the dialysate drain line 250, and the replacement solution pump 261 disposed in the replacement solution line 260, and that measures amounts of liquids (the dialysate and the replacement solution) flowing in the CRRT system 200. In a case where the liquid shortage sensor 214 detects a shortage of the liquids, the controller 280 operates the clamp 215 to close the blood return line 210b, thereby preventing air bubbles from entering the ECMO system 100.

Furthermore, in order to monitor the operation state of the ECMO system 100, the controller 280 acquires a measurement value from the flowmeter 114 and monitors the circuit internal pressure of the ECMO system 100 based on the measurement value of the pressure gauge P1 or the pressure gauge P3. Based on the measurement value from the flowmeter 114 and the measurement value from the pressure gauge P1 or the pressure gauge P3, the controller 280 grasps occurrence of a problem, such as unstable circulation in the ECMO system 100. The controller 280 includes a moisture removal controller 281, a circulation controller 282, and a switch controller 283, and performs control to maintain circulation of the blood in the CRRT blood circuit 210 in accordance with a degree of severeness of a problem occurring in the ECMO system 100.

The controller 280 determines that some problem has occurred in the ECMO system 100 in a case where a rate of change of the flow rate in the ECMO system 100 measured by the flowmeter 114 exceeds a predetermined threshold. In response to the controller 280 determining that some problem has occurred, the moisture removal controller 281 performs control so that the concentration of the blood circulating in the CRRT blood circuit 210 is maintained at a predetermined concentration. Specifically, the moisture removal controller 281 controls the flow rate of the drain pump 251 so as to keep the concentration of the blood circulating in the CRRT blood circuit 210 constant. For example, the moisture removal controller 281 keeps the concentration of the circulating blood constant by making the flow rate of the drain pump 251 substantially equal to the flow rate of the replacement solution pump 261. The moisture removal controller 281 stops the replacement solution pump 261 and the drain pump 251 to stop the blood purifier 230 from performing the blood purification and thereby keeps the concentration of the circulating blood constant. As a result, the blood in the CRRT blood circuit 210 can be prevented from concentrating, whereby formation of a thrombus can be suppressed.

In a case where a rate of change of the flow rate in the ECMO system 100 measured by the flowmeter 114 exceeds the predetermined threshold while the pressure measured by the pressure gauge is within a predetermined range, the controller 280 determines that the problem that has occurred is a minor problem. For example, in a case where there is a problem that the blood drainage amount decreases slightly due to partial blockage of the blood introduction port of the blood drainage cannula 111 or a similar factor while no malfunction has occurred in the ECMO blood pump 120 or the oxygenator 130, the flow rate in the ECMO system 100 measured by the flowmeter 114 decreases, whereas the pressure measured by the pressure gauge does not greatly change (and is maintained within a predetermined range). In this case, the controller 280 determines that the problem that has occurred is a minor problem.

In response to the controller 280 determining that such a minor problem has occurred, the circulation controller 282 reduces the flow rate of the blood purification pump 220 to a predetermined flow rate. In this case, at least part of the liquid flowing through the blood return line 210b is caused to flow again into the blood drainage line 210a via a portion of the ECMO system 100 (a portion of the blood drainage line 110a). That is, at least part of the blood (liquid) is recirculated in the CRRT blood circuit 210. Here, the predetermined flow rate refers to flow rates at which formation of a thrombus can be suppressed in the CRRT blood circuit 210. Accordingly, since the circulation of blood is not stopped even at the connection between the ECMO system 100 and the CRRT system 200, when the problem in the ECMO system 100 is solved, the cooperation with the CRRT system 200 can be promptly restarted.

In a case where a rate of change of the flow rate in the ECMO system 100 measured by the flowmeter 114 exceeds the predetermined threshold and the pressure measured by the pressure gauge exceeds the predetermined range, the controller 280 determines that a severe problem has occurred in the ECMO system 100. For example, when the performance of the ECMO blood pump 120 has decreased significantly, the flow rate in the ECMO system 100 measured by the flowmeter 114 decreases significantly (the flow rate decreases with the rate of change of the flow rate exceeding the predetermined threshold) and the pressure measured by the pressure gauge also decreases significantly (decreases below the predetermined range). In a case where the oxygenator 130 is clogged, the flow rate in the ECMO system 100 measured by the flowmeter 114 disposed downstream of the oxygenator 130 decreases significantly (the flow rate decreases with the rate of change of the flow rate exceeding the predetermined threshold), and the pressure measured by the pressure gauge increases significantly (increases beyond the predetermined range). In this case, the controller 280 determines that the problem that has occurred is a severe problem.

In response to the controller 280 determining that the problem is a severe problem, the switch controller 283 performs control to close the flow path to the ECMO system 100 and switch to the bypass line 270 to recirculate all the blood (liquid) in the CRRT blood circuit 210. Specifically, the switch controller 283 switches the second flow path switch 272 by closing the clamp 215 of the blood return line 210b and opening the bypass clamp 270b of the bypass line 270, thereby switching the flow path for the blood (liquid) flowing through the blood return line 210b to the bypass line 270. In addition, the switch controller 283 switches the first flow path switch 271 by closing the clamp 212 of the blood drainage line 210a and opening the bypass clamp 270a of the bypass line 270, thereby stopping the inflow of the blood (liquid) from the ECMO system 100 to the blood drainage line 210a and causing the blood (liquid) flowing through the bypass line 270 to flow into the blood drainage line 210a.

Each of the lines in the ECMO system 100 and the CRRT system 200 is mainly composed of a flexible tube through which liquid can flow.

According to the ECMO system 100 and the CRRT system 200 described above, the blood drawn from a vein of the subject (patient) flows into the blood drainage line 110a of the ECMO system 100, and part of the blood flows into the blood drainage line 210a of the CRRT system 200 at a predetermined flow rate (flow rate of the blood purification pump 220), and the remainder of the blood is sent to the ECMO blood pump 120. The part of the blood sent to the blood drainage line 210a of the CRRT system 200 is introduced into the blood purifier 230 at the predetermined flow rate. The blood purified by the blood purifier 230 is replenished with the replacement solution from the replacement solution line 260 in accordance with the amount of removed moisture, and then sent to the blood return line 210b.

The blood returned from the blood return line 210b to the blood drainage line 110a of the ECMO system 100 is sent to the ECMO blood pump 120 together with the blood flowing through the blood drainage line 110a. Thereafter, all the blood is sent to the oxygenator 130, where it is oxygenated and carbon dioxide is removed from it. The blood let out of the oxygenator 130 is returned to the patient's artery or vein via the blood return line 110c and the blood return cannula 112.

In a case where some problem has occurred in the ECMO system 100, the moisture removal controller 281 controls the flow rate of the drain pump 251 so that the concentration of the blood circulating in the CRRT blood circuit 210 is maintained constant. As result, the blood in the CRRT blood circuit 210 is prevented from concentrating, and formation of a thrombus is suppressed.

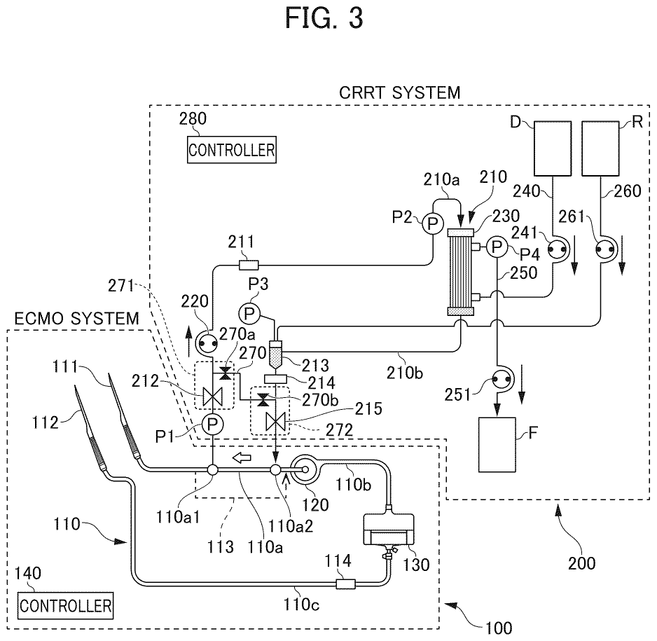

As illustrated in FIG. 3, in a case where the problem that has occurred in the ECMO system 100 is a minor problem, at least part of the blood is recirculated through the blood drainage line 210a, the blood return line 210b, and a portion of the ECMO system 100. At this time, the flow rate of the drain pump 251 is controlled to maintain the concentration of the circulating blood constant. Therefore, even if the recirculation is performed in the CRRT system 200 during a waiting time for the recovery of the ECMO system 100, the blood in the CRRT blood circuit 210 can be prevented from concentrating, thereby making it possible to suppress the formation of a thrombus. In addition, since the circulation of the blood is not stopped even at the connection between the ECMO system 100 and the CRRT system 200, the cooperation with the CRRT system 200 is promptly restarted once the problem in the ECMO system 100 is solved.

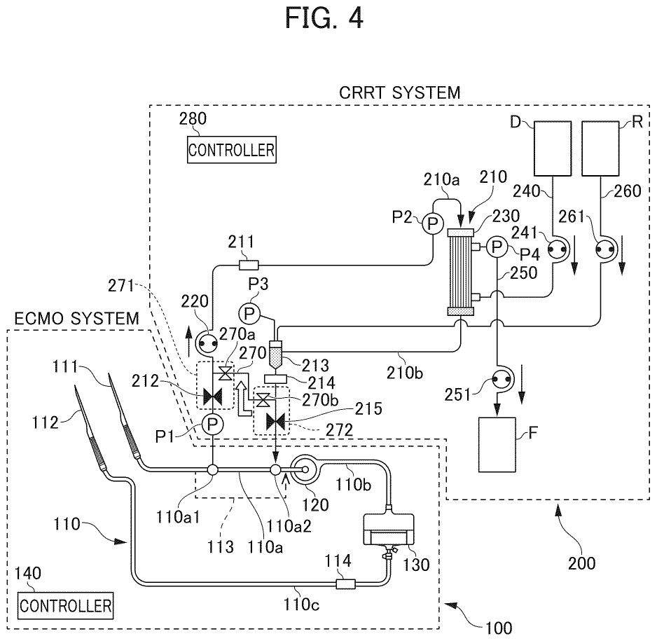

As illustrated in FIG. 4, in a case where a severe problem has occurred in the ECMO system 100, all the blood is recirculated through the blood drainage line 210a, the blood return line 210b, and the bypass line 270. At this time, the flow rate of the drain pump 251 is controlled to maintain the concentration of the circulating blood constant. Therefore, the formation of a thrombus in the CRRT blood circuit 210 is suppressed. In addition, since the recirculation is performed in a state in which the CRRT system 200 is disconnected from the ECMO system 100 during a waiting time for the recovery of the ECMO system 100, the recovery operation of the ECMO system 100 is facilitated.

### Second Embodiment

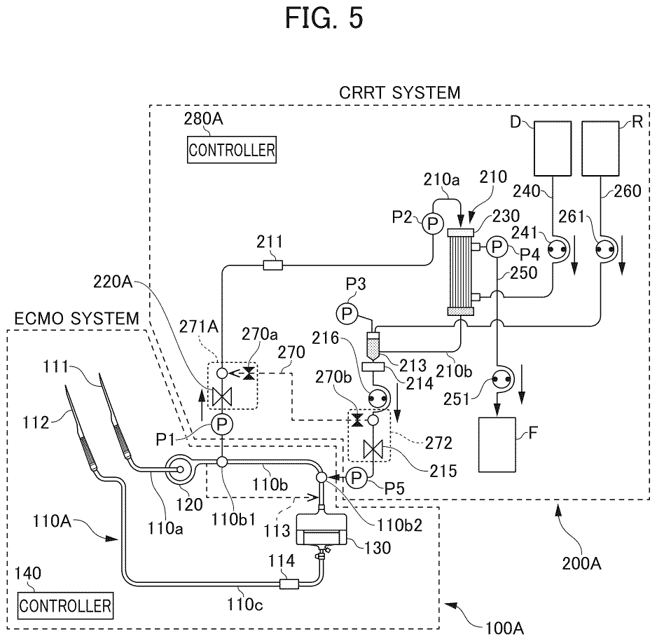

A second embodiment will be described in detail with reference to FIGS. 5 to 8. An ECMO system 100A according to the second embodiment is different from the first embodiment in that the first branch portion, the second branch portion, and the bypass line are provided not to a blood drainage line 110a but to a connection line 110b. A blood purification system 200A according to the second embodiment is different from the first embodiment in that a flow rate adjustment clamp is provided as a flow rate adjuster instead of the blood purification pump, and that a blood return pump is provided to a blood return line. Therefore, components similar to those described in the first embodiment are denoted by the same reference signs, and description thereof will be omitted. The differences from the first embodiment will be described.

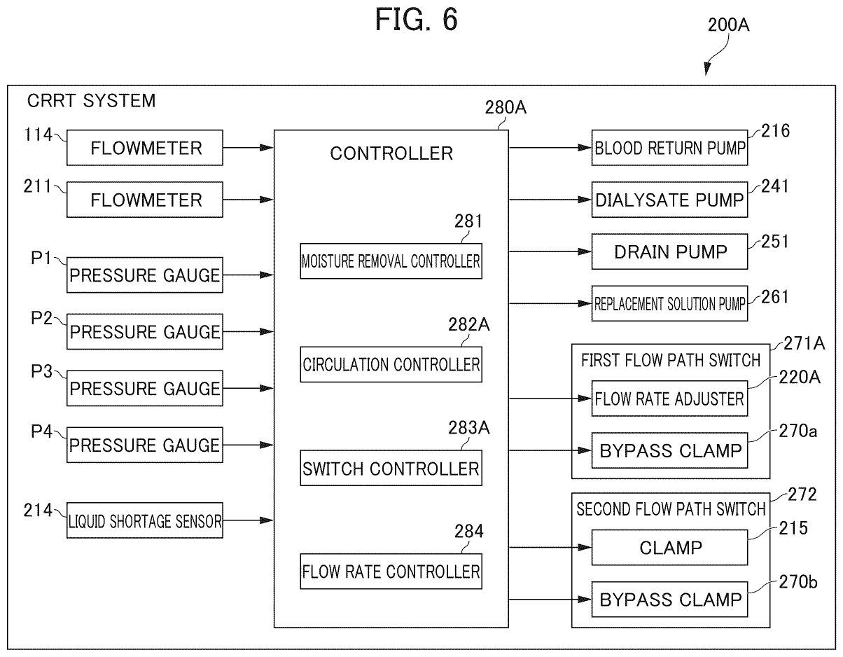

FIG. 5 is a diagram schematically illustrating the configuration of the ECMO system 100A and that of the CRRT system 200A according to the second embodiment of the present invention. FIG. 6 is a block diagram of the CRRT system 200A.

### ECMO System

As illustrated in FIG. 5, the ECMO system 100A includes an ECMO blood circuit 110A, an ECMO blood pump 120, an oxygenator 130, and a controller 140. The ECMO blood circuit 110A is a circuit for extracorporeally circulating the blood of a patient, and includes a blood drainage line 110a, a connection line 110b, and a blood return line 110c. The blood drainage line 110a has one end connected to a blood drainage cannula 111 and the other end connected to the ECMO blood pump 120. The connection line 110b has one end connected to the ECMO blood pump 120 and the other end connected to the oxygenator 130.

The connection line 110 b has a first branch portion 110b1 and a second branch portion 110b2. A connector such as a three-way stopcock is attached to each of the first branch portion 110b1 and the second branch portion 110b2. In the present embodiment, the connection line 110b is provided with a bypass line 113 that bypasses the first branch portion 110b1 and the second branch portion 110b2.

### CRRT System

As illustrated in FIGS. 5 and 6, the CRRT system 200A includes a CRRT blood circuit 210, a flow rate adjustment clamp 220A as a flow rate adjuster, a blood return pump 216, a blood purifier 230, a dialysate supply line 240, a dialysate drain line 250, a replacement solution line 260, a bypass line 270, and a controller 280A.

The CRRT blood circuit 210 is a circuit for circulating the drawn blood and includes a blood drainage line 210a and a blood return line 210b. The blood drainage line 210a has one end (upstream end) connected to the first branch portion 110b1 provided to the connection line 110b of the ECMO system 100A, and the other end (downstream end) connected to the blood purifier 230. The blood drainage line 210a is provided with the flow rate adjustment clamp 220A. A pressure gauge P1 is attached to a point upstream of the flow rate adjustment clamp 220A, and a pressure gauge P2 is attached to a point downstream of the flow rate adjustment clamp 220A. A flowmeter 211 is attached to the blood drainage line 210a to monitor whether the blood is being sent toward the blood purifier 230 at a normal flow rate.

The blood return line 210b has one end (upstream end) connected to the blood purifier 230, and the other end (downstream end) connected to the second branch portion 110b2 provided to the connection line 110b of the ECMO system 100.

The blood return line 210b has a drip chamber 213, a liquid shortage sensor 214, a blood return pump 216, a clamp 215, and a pressure gauge P5 that are attached in this order from the upstream side, and the drip chamber 213 has a pressure gauge P3 attached thereto. A known roller pump is used as the blood return pump 216. The drip chamber 213 stores therein a certain amount of the blood in order to remove air bubbles mixed in the blood, coagulated blood, and the like. The drip chamber 213 is also used as a pressure buffer for buffering a difference in flow rate between the flow rate adjustment clamp 220A and the blood return pump 216. Due to this configuration, even when the blood return line 210b is connected to a positive pressure portion (a point downstream of the ECMO blood pump 120) in the ECMO system 100A, the CRRT system 200A can be operated at a low positive pressure. The pressure gauge P3 is used as a buffer pressure gauge for measuring the pressure of the pressure buffer. The pressure gauge P5 reflects a pressure at the connection between the blood return line 210b and the ECMO blood circuit 110A, and therefore, it can be used to monitor a circuit internal pressure of the ECMO blood circuit 110A.

The flow rate adjustment clamp 220A is an adjustment clamp whose degree of opening is adjustable, and connects the blood drainage line 210a to the positive pressure portion (a point downstream of the ECMO blood pump 120) in the ECMO system 100A, thereby allowing the blood to be drawn at a predetermined flow rate. The drawn blood is sent to the blood purifier 230 through the blood drainage line 210a, and then returned to the ECMO system 100A through the blood return line 210b. In the present embodiment, an example in which the flow rate adjustment clamp is used as the flow rate adjuster 220A is illustrated, but a known roller pump may be used as the flow rate adjuster 220A. In the case where such a roller pump is used as the flow rate adjuster 220A, the blood can be drawn at a predetermined flow rate regardless of whether the blood drainage line 210a is connected to the positive pressure portion or a negative pressure portion (a point upstream of the ECMO blood pump 120) in the ECMO system 100A.

The bypass line 270 connects the blood drainage line 210a to the blood return line 210b and is used to disconnect the CRRT system 200A from the ECMO system 100A. The bypass line 270 is connected to a point downstream of the flow rate adjustment clamp 220A in the blood drainage line 210a and is connected to a point downstream of the blood return pump 216 in the blood return line 210b. The bypass line 270 has a bypass clamp 270a provided near the connection with the blood drainage line 210a and a bypass clamp 270b provided near the connection with the blood return line 210b. The bypass clamp 270a and the flow rate adjustment clamp 220A provided to the blood drainage line 210a together constitute a first flow path switch 271A. The bypass clamp 270b and the clamp 215 provided to the blood return line 210b together constitute a second flow path switch 272. In the present embodiment, the first flow path switch 271A is constituted by the flow rate adjuster 220A and the bypass clamp 270a, and the second flow path switch 272 is constituted by the two clamps, but each of the first and second flow path switches may be constituted by a three-way stopcock or the like.

The controller 280A is constituted by an information processing apparatus (computer) and executes a control program to drive the pumps included in the CRRT system 200A to control the blood flow rate, the dialysate amount, and the drainage amount for the blood purifier 230, thereby performing continuous hemodiafiltration (CHDF). The controller 280A may control and drive the pumps based on flow rates measured by a metering unit (not shown) that monitors a flow rate of each of the dialysate pump 241 disposed in the dialysate supply line 240, the drain pump 251 disposed in the dialysate drain line 250, and the replacement solution pump 261 disposed in the replacement solution line 260, and that measures amounts of liquids (the dialysate and the replacement solution) flowing in the CRRT system 200A. In a case where the liquid shortage sensor 214 detects a shortage of the liquids, the controller 280A operates the clamp 215 to close the blood return line 210b, thereby preventing air bubbles from entering the ECMO system 100A.

Furthermore, in order to monitor the operation state of the ECMO system 100A, the controller 280A acquires a measurement value from the flowmeter 114 and monitors the circuit internal pressure of the ECMO system 100A based on the measurement value of the pressure gauge P1 or the pressure gauge P5. Based on the measurement value from the flowmeter 114 and the measurement value from the pressure gauge P1 or the pressure gauge P5, the controller 280A grasps occurrence of a problem, such as unstable circulation in the ECMO system 100A. The controller 280A includes a moisture removal controller 281, a circulation controller 282A, and a switch controller 283A, and performs control to maintain circulation of the blood in the CRRT blood circuit 210 in accordance with a degree of severeness of a problem occurring in the ECMO system 100A. The controller 280A further includes a flow rate controller 284 that adjusts the flow rate of the flow rate adjustment clamp 220A and the flow rate of the blood return pump 216 so as to control a value measured by the buffer pressure gauge P3 to be within a predetermined range.

The controller 280A determines that some problem has occurred in the ECMO system 100A in a case where a rate of change of the flow rate in the ECMO system 100A measured by the flowmeter 114 exceeds a predetermined threshold. In response to the controller 280A determining that some problem has occurred, the moisture removal controller 281 controls the CRRT system 200A so that the concentration of the blood circulating in the CRRT blood circuit 210 is maintained at a predetermined concentration. Specifically, the moisture removal controller 281 controls the flow rate of the drain pump 251 so as to maintain the concentration of the blood circulating in the CRRT blood circuit 210 constant.

In a case where a rate of change of the flow rate in the ECMO system 100A measured by the flowmeter 114 exceeds the predetermined threshold while the pressure measured by the pressure gauge is within a predetermined range, the controller 280A determines that the problem that has occurred is a minor problem.

In response to the controller 280A determining that the problem is a minor problem, the circulation controller 282A adjusts the degree of opening of the flow rate adjustment clamp 220A and the output of the blood return pump 216 to reduce the flow rate of the blood flowing through the CRRT blood circuit 210 to a predetermined flow rate. In this case, at least part of the liquid flowing through the blood return line 210b is caused to flow again into the blood drainage line 210a via a portion of the ECMO system 100A (a portion of the blood drainage line 110a). That is, at least part of the blood (liquid) is recirculated in the CRRT blood circuit 210.

In a case where a rate of change of the flow rate in the ECMO system 100A measured by the flowmeter 114 exceeds the predetermined threshold and the pressure measured by the pressure gauge exceeds the predetermined range, the controller 280A determines that a severe problem has occurred in the ECMO system 100A.

In response to the controller 280A determining that the problem is a severe problem, the switch controller 283A performs control to close the flow path to the ECMO system 100 and switch to the bypass line 270 to recirculate all the blood (liquid) in the CRRT blood circuit 210. Specifically, the switch controller 283 switches the second flow path switch 272 by closing the clamp 215 of the blood return line 210b and opening the bypass clamp 270b of the bypass line 270, thereby switching the flow path for the blood (liquid) flowing through the blood return line 210b to the bypass line 270. In addition, the switch controller 283A switches the first flow path switch 271A by setting the degree of opening of the flow rate adjustment clamp 220A of the blood drainage line 210a to 0 and opening the bypass clamp 270a of the bypass line 270, thereby stopping the inflow of the blood (liquid) from the ECMO system 100A to the blood drainage line 210a and causing the blood (liquid) flowing through the bypass line 270 to flow into the blood drainage line 210a.

According to the ECMO system 100A and the CRRT system 200A described above, the blood drawn from a vein of the subject (patient) flows into the blood drainage line 110a of the ECMO system 100A, and part of the blood flows into the blood drainage line 210a of the CRRT system 200A at a predetermined flow rate (flow rate of the flow rate adjuster 220A) from the connection line 110b, and the remainder of the blood is sent to the oxygenator 130. The part of the blood sent to the blood drainage line 210a of the CRRT system 200A is introduced into the blood purifier 230 at the predetermined flow rate. Here, the flow rate adjustment clamp 220A functions as a liquid sending means for sending the blood to the blood purifier 230, and also functions as a pressure partition that prevents a high positive pressure transmitted from the ECMO blood circuit 110A from being transmitted to the blood purifier 230. The blood purified by the blood purifier 230 is replenished with the replacement solution from the replacement solution line 260 in accordance with the amount of removed moisture, and then sent to the blood return line 210b.

In the blood return line 210b, the drip chamber 213 as a pressure buffer stores therein the blood within a predetermined range. The storage amount can be controlled by adjusting the set flow rate of the blood return pump 216. Specifically, the flow rate of the blood sent from the downstream end of the blood purifier 230 is slightly lower than the set flow rate of the flow rate adjustment clamp 220A by the amount of removed moisture. Therefore, in a case where the internal pressure (the measurement value of the pressure gauge P3) of the drip chamber 213 as the pressure buffer exceeds a predetermined range, it is suitable to make the set flow rate of the blood return pump 216 higher than the set flow rate of the flow rate adjustment clamp 220A. In a case where the internal pressure (the measurement value of the pressure gauge P3) of the drip chamber 213 as the pressure buffer falls below the predetermined range, it is suitable to make the set flow rate of the blood return pump 216 lower than the set flow rate of the flow rate adjustment clamp 220A. By adjusting the amount of liquid stored in the drip chamber 213 in this manner, the measurement value of the pressure gauge P3 can be set within the predetermined range. As a result, the portion upstream of the blood return pump 216 in the blood return line 210b can be set to a low positive pressure, thereby making it possible to operate the CRRT system 200A at a low positive pressure. Since the blood return line 210b is provided with the blood return pump 216, the circuit internal pressure of the portion downstream of the blood return pump 216 can be raised and the blood can be sent to the positive pressure portion of the ECMO system 100A (the portion downstream of the ECMO blood pump 120). Here, the blood return pump 216 functions as a liquid sending means for sending the blood to the positive pressure portion of the ECMO system 100A, and also functions as a pressure partition that sets the portion upstream thereof to a low positive pressure and the portion downstream thereof to a high positive pressure.

The blood returned from the blood return line 210b to the connection line 110b of the ECMO system 100A is sent, together with the blood flowing through the connection line 110b, to the oxygenator 130, where all the blood is oxygenated and carbon dioxide is removed. The blood let out of the oxygenator 130 is returned to the patient's artery or vein via the blood return line 110c and the blood return cannula 112.

In a case where some problem has occurred in the ECMO system 100A, the moisture removal controller 281 controls the flow rate of the drain pump 251 so that the concentration of the blood circulating in the CRRT blood circuit 210 is maintained constant. As result, the blood in the CRRT blood circuit 210 is prevented from concentrating, and formation of a thrombus is suppressed.

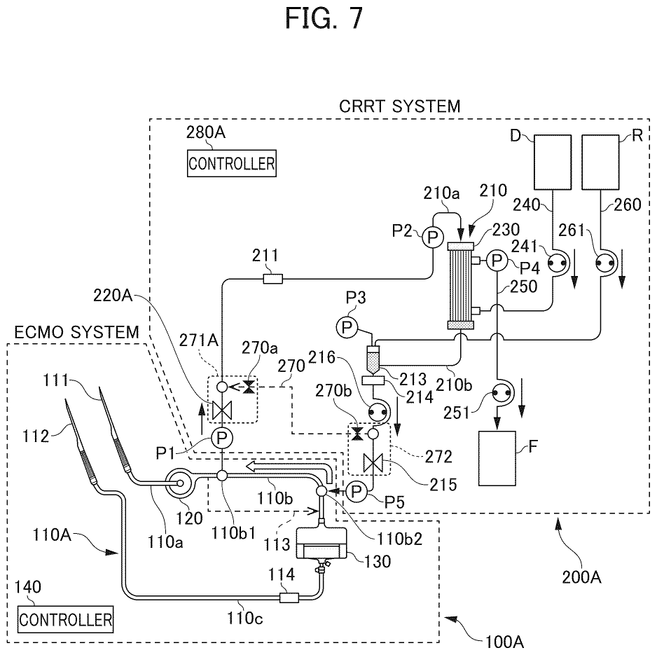

As illustrated in FIG. 7, in a case where the problem that has occurred in the ECMO system 100A is a minor problem, at least part of the blood is recirculated through the blood drainage line 210a, the blood return line 210b, and a portion of the ECMO system 100. At this time, the flow rate of the drain pump 251 is controlled to maintain the concentration of the circulating blood constant. Therefore, even if the recirculation is performed in the CRRT system 200A during a waiting time for the recovery of the ECMO system 100, the blood in the CRRT blood circuit 210 can be prevented from concentrating, thereby making it possible to suppress the formation of a thrombus. In addition, since the circulation of the blood is not stopped even at the connection between the ECMO system 100A and the CRRT system 200A, the cooperation with the CRRT system 200A is promptly restarted once the problem in the ECMO system 100A is solved.

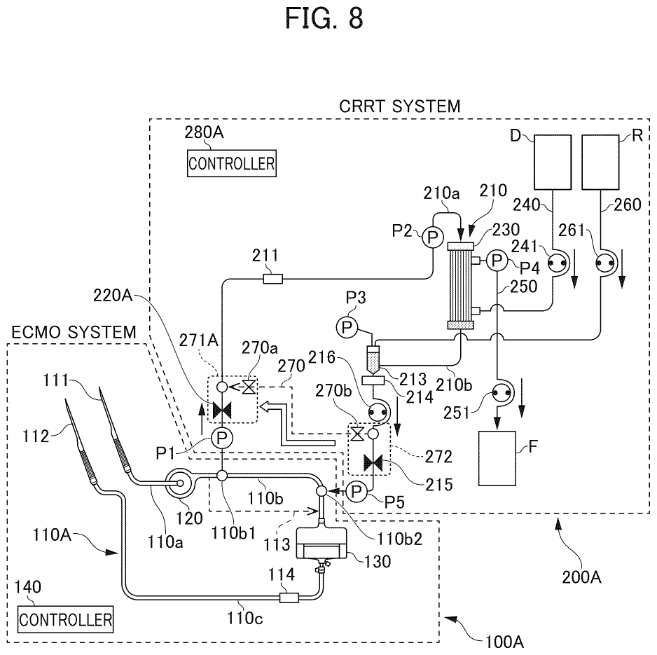

As illustrated in FIG. 8, in a case where a severe problem has occurred in the ECMO system 100A, all the blood is recirculated through the blood drainage line 210a, the blood return line 210b, and the bypass line 270. At this time, the flow rate of the drain pump 251 is controlled to maintain the concentration of the circulating blood constant. Therefore, the formation of a thrombus in the CRRT blood circuit 210 is suppressed. In addition, since the recirculation is performed in a state in which the CRRT system 200A is disconnected from the ECMO system 100A during a waiting time for the recovery of the ECMO system 100A, the recovery operation of the ECMO system 100A is facilitated.

According to the second embodiment, the blood purification system 200A includes the pressure buffer (drip chamber 213) that is provided to a point upstream of the blood return pump 216 in the blood return line 210b and is capable of storing a predetermined amount of liquid, and the buffer pressure gauge P3 that measures a pressure of the pressure buffer (drip chamber 213). The controller 280A includes the flow rate controller 284 that adjusts the degree of opening of the flow rate adjustment clamp 220A and the output of the blood return pump 216 so as to control the measured value of the buffer pressure gauge P3 to be within a predetermined range. Due to this configuration, even in a case where the blood return line 210b of the blood purification system 200A is connected to the positive pressure portion of the ECMO system 100A, the blood purification system 200A can be operated at a low positive pressure.

### Third Embodiment

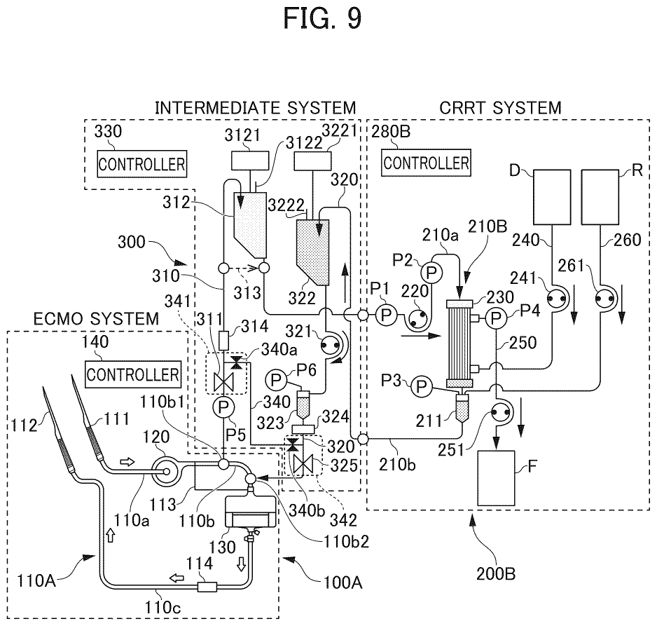

A third embodiment will be described in detail with reference to FIGS. 9 to 12. FIG. 9 is a diagram schematically illustrating a configuration of an ECMO system 100A, that of a CRRT system 200B, and that of an intermediate system 300 according to the third embodiment of the present invention.

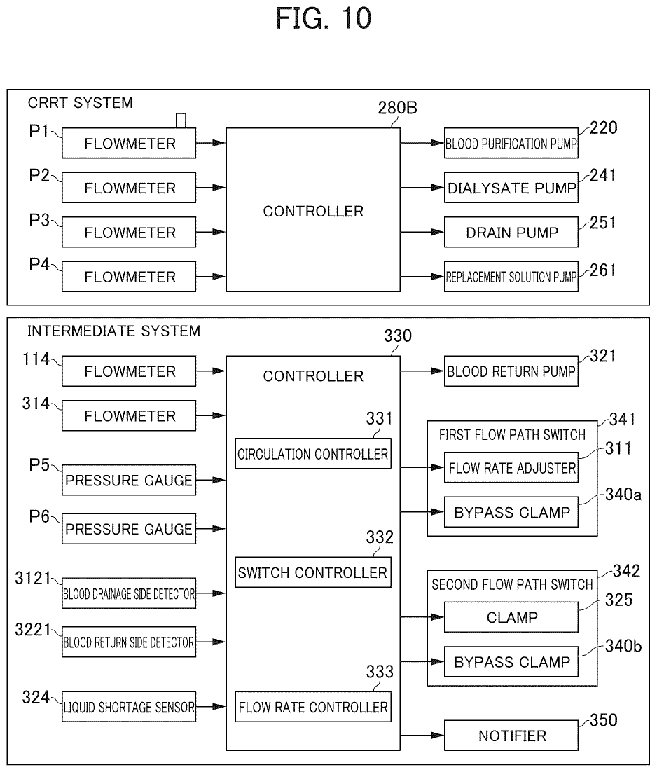

Since the ECMO system 100A is the same as that described in the second embodiment, description thereof will be omitted. FIG. 10 is a block diagram of the CRRT system 200B and the intermediate system 300.

### CRRT System

As shown in FIGS. 9 and 10, the CRRT system 200B includes a CRRT blood circuit 210B, a blood purification pump 220, a blood purifier 230, a dialysate supply line 240, a dialysate drain line 250, a replacement solution line 260, and a controller 280B.

The CRRT blood circuit 210B is a circuit for circulating drawn blood and includes a blood drainage line 210a and a blood return line 210b. The blood drainage line 210a has one end (upstream end) connected to a downstream end of an intermediate blood drainage line 310 of the intermediate system 300, which will be described later, and the other end (downstream end) connected to the blood purifier 230. The blood drainage line 210a is provided with the blood purification pump 220. A pressure gauge P1 is attached to a point upstream of the blood purification pump 220, and a pressure gauge P2 is attached to a point downstream of the blood purification pump 220. The blood return line 210b has one end (upstream end) connected to the blood purifier 230, and the other end (downstream end) connected to an upstream end of an intermediate blood return line 320 of the intermediate system 300, which will be described later.

The blood purification pump 220 draws the blood from the ECMO system 100A via the intermediate blood drainage line 310. The drawn blood is sent to the blood purifier 230 through the blood drainage line 210a, and then returned to the ECMO system 100A through the blood return line 210b and the intermediate blood return line 320.

The controller 280B is constituted by an information processing apparatus (computer) and executes a control program to drive the pumps included in the CRRT system 200B to control a blood flow rate, a dialysate amount, and a drainage amount for the blood purifier 230, thereby performing continuous hemodiafiltration (CHDF). The controller 280B may control and drive the pumps based on a flow rate measured by a metering unit (not shown) that monitors the flow rate of each of a dialysate pump 241 disposed in the dialysate supply line 240, a drain pump 251 disposed in the dialysate drain line 250, and a replacement solution pump 261 disposed in the replacement solution line 260 and that measures amounts of liquids (the dialysate and the replacement solution) flowing in the CRRT system 200B.

### Intermediate System

As illustrated in FIGS. 9 and 10, the intermediate system 300 includes the intermediate blood drainage line 310, a flow rate adjuster 311, the intermediate blood return line 320, a blood return pump 321, a blood drainage side pressure buffer 312, a blood drainage side detector 3121, a blood return side pressure buffer 322, a blood return side detector 3221, a controller 330, a bypass line 340, and a notifier 350 (see FIG. 10).

The intermediate blood drainage line 310 is a line through which part of the blood flowing through a connection line 110b of the ECMO system 100A is drawn and sent to the CRRT system 200B. The intermediate blood drainage line 310 has one end (upstream end) connected to a first branch portion 110b1 provided to the connection line 110b of the ECMO system 100A, and the other end (downstream end) connected to an upstream end of the CRRT system 200B. The intermediate blood drainage line 310 is provided with the flow rate adjuster 311. A pressure gauge P5 is attached to a point upstream of the flow rate adjuster 311. In addition, a flowmeter 314 is attached to the intermediate blood drainage line 310 to monitor whether the blood is being sent to the connection with the CRRT system 200 (the upstream end of the blood drainage line 210a) at a normal flow rate. In the present embodiment, an ultrasonic flowmeter is used as the flowmeter 314. An optical flow meter or the like may be used as the flowmeter 314.

The flow rate adjuster 311 adjusts the flow rate of the blood flowing through the intermediate blood drainage line 310. The flow rate adjuster 311 is attached to a point close to the upstream end of the intermediate blood drainage line 310. Accordingly, in a case where the blood flow needs to be stopped by closing the flow rate adjuster 311, it is possible to reduce an amount of the blood to be discarded because of coagulation. The flow rate adjuster 311 is constituted by a flow rate adjustment clamp whose degree of opening can be adjusted. As the flow rate adjuster 311, a roller pump may be used instead of the flow rate adjustment clamp. Adjusting the flow rate of the blood flowing through the intermediate blood drainage line 310 by the flow rate adjuster 311 makes it possible to adjust a circuit internal pressure of a portion downstream of the flow rate adjuster 311.

The blood drainage side pressure buffer 312 is provided at a point downstream of the flow rate adjuster 311 in the intermediate blood drainage line 310 and can store therein a predetermined amount of liquid. In the present embodiment, a reservoir formed of a soft bag is used as the blood drainage side pressure buffer 312. Preferably, the blood drainage side pressure buffer 312 (reservoir) has an inlet and an outlet for the blood (liquid) in its lower portion so that the inlet and the outlet are filled with the blood (liquid). For example, two openings are suitably formed as the inlet and the outlet in the lower portion of the reservoir. This configuration makes it possible to reduce the risk of sending air to the intermediate blood drainage line 310 downstream of the reservoir when the amount of the liquid stored in the reservoir rapidly decreases for some reason. In addition, a deaeration line 3122 (see FIG. 9) may be provided in an upper portion of the reservoir. The deaeration line 3122 is in principle closed during operation and is used when a liquid level is checked during priming, and when releasing air accumulated in the reservoir during operation. Furthermore, a warming mechanism (not shown) may be provided to the blood drainage side pressure buffer 312. The warming mechanism can warm the blood to be returned to the ECMO blood circuit 110. As the blood drainage side pressure buffer 312, a small container (for example, a pillow) made of a soft material may be used instead of the reservoir.

The intermediate blood drainage line 310 may be provided with a bypass line 313 for bypassing the blood drainage side pressure buffer 312. The connection between the bypass line 313 and the intermediate blood drainage line 310 is constituted by, for example, a Y-connector (not shown). Provision of the bypass line 313 allows the user to choose whether to use the blood drainage side pressure buffer 312 to perform pressure buffering, or to use the bypass line 313 to omit the pressure buffering using the blood drainage side pressure buffer 312 when the user operates the intermediate system 300. When the blood drainage side pressure buffer 312 is not used, a priming volume can be reduced accordingly.

The blood drainage side detector 3121 is attached to the blood drainage side pressure buffer 312 and detects a storage state of the blood drainage side pressure buffer 312. In the present embodiment, a weight meter is used as an example of the blood drainage side detector 3121 to measure the weight (storage state) of the liquid stored in the blood drainage side pressure buffer 312 (reservoir). In a case where an air layer is provided in the reservoir, a pressure gauge may be used as the blood drainage side detector 3121 to measure an internal pressure that varies in accordance with the storage state of the reservoir. In a case where a small container such as a pillow is used as the blood drainage side pressure buffer 312, a pressure gauge is suitably used as the blood drainage side detector 3121 to detect expansion and contraction that occur in accordance with an internal pressure of the small container.

The intermediate blood return line 320 has one end (downstream end) connected to a second branch portion 110b2 provided to the connection line 110b of the ECMO system 100A, and the other end (upstream end) connected to a downstream end of the CRRT blood circuit 210 (a blood return line 210b). The intermediate blood return line 320 has the blood return side pressure buffer 322 and the blood return pump 321 in this order from the upstream side. Furthermore, the intermediate blood return line 320 has a drip chamber 323, a liquid shortage sensor 324, and a clamp 325 that are sequentially attached to a portion downstream of the blood return pump 321. A pressure gauge P6 is attached to the drip chamber 323. The drip chamber 323 stores therein a certain amount of the blood in order to remove air bubbles mixed in the intermediate blood return line 320, coagulated blood, and the like. The clamp 325 is attached to a point close to the downstream end of the intermediate blood return line 320. The pressure gauge P6 measures a circuit internal pressure of a portion downstream of the blood return pump 321 in the intermediate blood return line 320.

The blood return pump 321 is a pump for pumping the blood flowing through the intermediate blood return line 320 to a positive pressure portion of the ECMO system 100A. A known roller pump can be used as the blood return pump 321.

The blood return side pressure buffer 322 is provided at a point upstream of the blood return pump 321 in the intermediate blood return line 320 and can store therein a predetermined amount of liquid. In the present embodiment, a reservoir formed of a soft bag is used as the blood return side pressure buffer 322, similarly to the blood drainage side pressure buffer 312.

The blood return side detector 3221 is attached to the blood return side pressure buffer 322 and detects a storage state of the blood return side pressure buffer 322. The blood return side detector 3221 may have the same configuration as that of the blood drainage side detector 3121.

The bypass line 340 is a line connecting the intermediate blood drainage line 310 to the intermediate blood return line 320 and is used to disconnect the CRRT system 200B and the intermediate system 300 from ECMO system 100A. The bypass line 340 is connected to a point downstream of the flow rate adjuster 311 in the intermediate blood drainage line 310 and is connected to a point between the liquid shortage sensor 324 and the clamp 325 in the intermediate blood return line 320. The bypass line 340 has a bypass clamp 340a in the vicinity of the connection with the intermediate blood drainage line 310 and a bypass clamp 340b in the vicinity of the connection with the intermediate blood return line 320. The bypass clamp 340a and the flow rate adjuster 311 provided to the intermediate blood drainage line 310 together constitute a first flow path switch 341. The bypass clamp 340b and the clamp 325 provided to the intermediate blood return line 320 together constitute a second flow path switch 342. In the present embodiment, the first flow path switch 341 is constituted by the flow rate adjuster 311 and the bypass clamp 340a, and the second flow path switch 342 is constituted by the two clamps, but each of the first and second flow path switches may be constituted by a three-way stopcock or the like.

The controller 330 is constituted by an information processing apparatus (computer) and drives and controls the pumps and the clamps included in the intermediate system 300 by executing a control program. The controller 330 further adjusts the flow rate in the intermediate blood drainage line 310 by controlling a degree of opening of the flow rate adjustment clamp as the flow rate adjuster 311 in accordance with the storage state (weight) detected by the blood drainage side detector 3121 (weight meter) so that the amount of the blood stored in the blood drainage side pressure buffer 312 (reservoir) is within a predetermined range. It is suitable to adjust and set the flow rate of the blood drawn from the connection line 110b based on the flow rate of the blood purification pump 220 as a reference. Furthermore, in a case where the liquid shortage sensor 324 detects a shortage of the liquid, the controller 330 operates the clamp 325 to close the intermediate blood return line 320, thereby preventing air bubbles from entering the ECMO system 100A.

Furthermore, in order to monitor an operation state of the ECMO system 100A, the controller 330 acquires a measurement value from the flowmeter 114 and monitors a circuit internal pressure of the ECMO system 100A based on a measurement value of the pressure gauge P5 or the pressure gauge P6. Based on the measurement value from the flowmeter 114 and the measurement value from the pressure gauge P5 or the pressure gauge P6, the controller 330 grasps occurrence of a problem, such as unstable circulation in the ECMO system 100A. The controller 330 includes a circulation controller 331 and a switch controller 332. In the present embodiment, the controller 330 determines that some problem has occurred in the ECMO system 100 in a case where a rate of change of the flow rate in the ECMO system 100 measured by the flowmeter 114 exceeds a predetermined threshold. The controller 330 causes the notifier 350 to notify the occurrence of the problem. Specifically, the controller 330 causes the notifier 350 to notify that some problem has occurred in the ECMO system 100 and that the blood purification by the blood purifier 230 needs to be stopped to maintain the concentration of the blood flowing through the CRRT blood circuit 210 constant.

In a case where a rate of change of the flow rate in the ECMO system 100A measured by the flowmeter 114 exceeds the predetermined threshold while the pressure measured by the pressure gauge is within a predetermined range, the controller 330 determines that the problem that has occurred is a minor problem. In this case, the circulation controller 331 reduces the flow rate of the blood purification pump 220 to a predetermined flow rate. At this time, at least part of the liquid flowing through the intermediate blood return line 320 flows again into the intermediate blood drainage line 310 via a portion of the ECMO system 100A (a portion of the connection line 110b). That is, the at least part of the blood (liquid) is recirculated in the intermediate system 300 and the CRRT blood circuit 210.

In a case where a rate of change of the flow rate in the ECMO system 100A measured by the flowmeter 114 exceeds the predetermined threshold and the pressure measured by the pressure gauge exceeds the predetermined range, the controller 330 determines that a severe problem has occurred in the ECMO system 100A. In this case, the switch controller 332 performs control to close the flow path to the ECMO system 100A and switch to the bypass line 340 to recirculate all the blood (liquid) through the intermediate blood drainage line 310, the CRRT blood circuit 210, the intermediate blood return line 320, and the bypass line 340. Specifically, the switch controller 332 switches the second flow path switch 342 by closing the clamp 325 of the intermediate blood return line 320 and opening the bypass clamp 340b of the bypass line 340, thereby switching the flow path for the blood (liquid) flowing through the intermediate blood return line 320 to the bypass line 340. At the same time, the switch controller 332 switches the first flow path switch 341 by setting the flow rate of the flow rate adjuster 311 of the intermediate blood drainage line 310 to 0 and opening the bypass clamp 340a of the bypass line 340, thereby stopping the inflow of the blood (liquid) from the ECMO system 100A to the intermediate blood drainage line 310 and causing the blood (liquid) flowing through the bypass line 340 to flow into the intermediate blood drainage line 310.

In the present embodiment, the controller 330 further includes a flow rate controller 333 that adjusts the flow rate of the flow rate adjuster 311 and the flow rate of the blood return pump 321 so as to control the weight of the liquid stored in the blood return side pressure buffer to be within a predetermined range.

Each of the lines in the ECMO system 100A, the CRRT system 200B, and the intermediate system 300 is mainly composed of a flexible tube through which a liquid can flow.

According to the ECMO system 100A, the CRRT system 200B, and the intermediate system 300 described above, the blood drawn from a vein of the subject (patient) flows into the blood drainage line 110a of the ECMO system 100A, and part of the blood flows into the intermediate blood drainage line 310 of the intermediate system 300 at a predetermined flow rate from the connection line 110b, and the remainder of the blood is sent to the oxygenator 130.

The blood sent to the intermediate system 300 is stored within a predetermined range in the blood drainage side pressure buffer 312. The storage amount can be controlled by adjusting the set flow rate of the flow rate adjuster 311. Specifically, the flow rate of the blood flowing out from the downstream side of the blood drainage side pressure buffer 312 is the set flow rate of the blood purification pump 220. Therefore, in a case where the storage amount of the blood drainage side pressure buffer 312 exceeds the predetermined range, it is suitable to make the set flow rate of the flow rate adjuster 311 lower than the set flow rate of the blood purification pump 220. In a case where the storage amount of the blood drainage side pressure buffer 312 is less than the predetermined range, it is suitable to make the set flow rate of the flow rate adjuster 311 higher than the set flow rate of the blood purification pump 220. In this way, the predetermined amount of the blood is stored in the blood drainage side pressure buffer 312, and as a result, the circuit internal pressure can be lowered by means of the blood drainage side pressure buffer 312 provided to the intermediate blood drainage line 310.

The blood stored in the blood drainage side pressure buffer 312 is introduced into the blood purifier 230 by the blood purification pump 220. Here, since the circuit internal pressure is set to a low positive pressure by the blood drainage side pressure buffer 312, the portion upstream of the blood purification pump 220 is at a low positive pressure. In the blood purifier 230, moisture and waste products are removed via a dialysis membrane, and the purified blood is let out of the blood purifier 230. The blood purified by the blood purifier 230 is replenished with the replacement solution from the replacement solution line 260 in accordance with the amount of removed moisture, and then sent to the intermediate blood return line 320 of the intermediate system 300.

In the intermediate blood return line 320, the blood return side pressure buffer 322 stores therein the blood within a predetermined range. The storage amount can be controlled by adjusting the set flow rate of the blood return pump 321. Specifically, the flow rate of the blood sent from the downstream end of the CRRT system 200B is slightly lower than the set flow rate of the blood purification pump 220 by the amount of removed moisture. Therefore, in a case where the storage amount of the blood return side pressure buffer 322 exceeds the predetermined range, it is suitable to make the set flow rate of the blood return pump 321 higher than the set flow rate of the blood purification pump 220. In a case where the storage amount of the blood return side pressure buffer 322 is less than the predetermined range, it is suitable to make the set flow rate of the blood return pump 321 lower than the set flow rate of the blood purification pump 220. In this way, the predetermined amount of the blood is stored in the blood return side pressure buffer 322, and as a result, the circuit internal pressure can be lowered by means of the blood return side pressure buffer 322 provided to the intermediate blood return line 320. Due to this configuration, the downstream end of the CRRT system 200B can be set to a low positive pressure, thereby making it possible to operate the CRRT system 200B at a low positive pressure. Since the intermediate blood return line 320 is provided with the blood return pump 321, the circuit internal pressure lowered by the blood return side pressure buffer 322 is raised and the blood can be sent to a positive pressure portion of the ECMO system 100A (a portion downstream of the ECMO blood pump 120). Here, the blood return pump 321 functions as a liquid sending means for sending the blood to the positive pressure portion of the ECMO system, and also functions as a pressure partition that sets the portion upstream thereof to a low positive pressure and the portion downstream thereof to a high positive pressure.

The blood returned from the intermediate blood return line 320 of the intermediate system 300 to the connection line 110b of the ECMO system 100A is sent, together with the blood flowing through the connection line 110b, to the oxygenator 130, where all the blood is oxygenated and carbon dioxide is removed. The blood let out of the oxygenator 130 is returned to the patient's artery or vein via the blood return line 110c and the blood return cannula 112.

In a case where some problem has occurred in the ECMO system 100A, the notifier 350 provides notification, and when the concentration of the blood flowing through the CRRT blood circuit 210 is maintained constant in response to the notification, the blood in the CRRT system 200B is prevented from concentrating, and formation of a thrombus is suppressed.

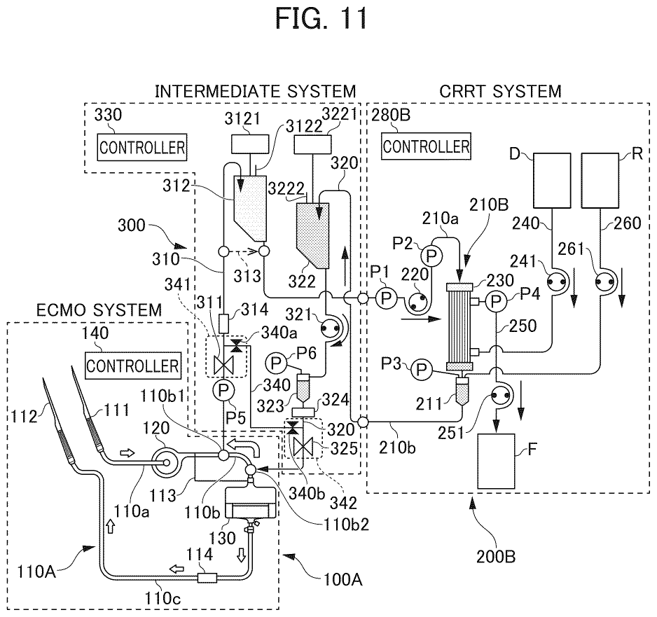

As illustrated in FIG. 11, in a case where the problem that has occurred in the ECMO system 100 is a minor problem, at least part of the blood is recirculated through the CRRT blood circuit 210, the intermediate blood drainage line 310, the intermediate blood return line 320, and a portion of the ECMO system 100A. Therefore, even if the recirculation is performed in the intermediate system 300 and the CRRT system 200B during a waiting time for the recovery of the ECMO system 100A, the blood can be prevented from concentrating, thereby making it possible to suppress the formation of a thrombus. In addition, since the circulation of the blood is not stopped even at the connection between the ECMO system 100 and the intermediate system 300, the cooperation with the CRRT system 200 is promptly restarted once the problem in the ECMO system 100 is solved.

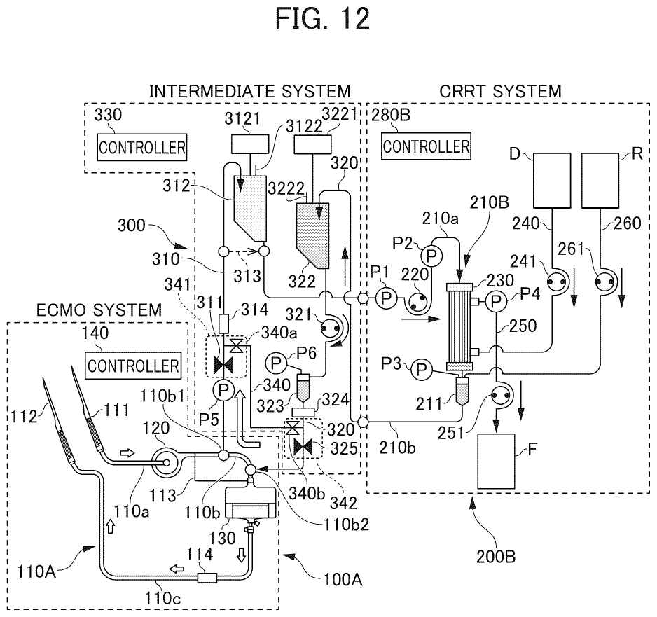

As illustrated in FIG. 12, in a case where a severe problem has occurred in the ECMO system 100A, all the blood is recirculated through the intermediate blood drainage line 310, the CRRT blood circuit 210, the intermediate blood return line 320, and the bypass line 340. At this time, the notifier 350 provides notification, and when the concentration of the blood flowing through the CRRT blood circuit 210 is maintained constant in response to the notification, the blood in the intermediate system 300 and the CRRT system 200B is prevented from concentrating, thereby making it possible to suppress the formation of a thrombus. In addition, since the recirculation is performed in a state in which the intermediate system 300 and the CRRT system 200A are disconnected from the ECMO system 100A during a waiting time for the recovery of the ECMO system 100A, the recovery operation of the ECMO system 100A is facilitated.

It should be noted that the present invention is not limited to the preferred embodiments of the CRRT system and the intermediate system described above and can be appropriately modified.

### EXPLANATION OF REFERENCE NUMERALS

100, 100A: Extracorporeal membrane oxygenation (ECMO) system
110, 110A: Blood circuit
110a: Blood drainage line
110b: Connection line
120: ECMO blood pump
130: Oxygenator
200, 200A, 200B: Continuous renal replacement therapy (CRRT) system
210, 210B: Blood circuit
210a: Blood drainage line
210b: Blood return line
220: Blood purification pump
230: Blood purifier
300: Intermediate system
310: Intermediate blood drainage line
311: Flow rate adjuster
312: Blood drainage side pressure buffer
320: Intermediate blood return line
321: Blood return pump
322: Blood return side pressure buffer
3121: Blood drainage side detector
3221: Blood return side detector

1. A blood purification system that is connected to an extracorporeal membrane oxygenation system including an ECMO blood pump and an oxygenator disposed downstream of the ECMO blood pump, the blood purification system comprising:
a blood drainage line having an upstream end that is connected to the extracorporeal membrane oxygenation system;
a flow rate adjuster provided to the blood drainage line and configured to adjust a flow rate in the blood drainage line;
a blood purifier connected to a downstream end of the blood drainage line;
a blood return line having an upstream end connected to the blood purifier and a downstream end that is connected to the extracorporeal membrane oxygenation system;
a flowmeter configured to measure a flow rate in the extracorporeal membrane oxygenation system; and
a controller,
the controller including
a moisture removal controller that maintains a concentration of liquid flowing through the blood purification system at a predetermined concentration in a case where a rate of change of the flow rate measured by the flowmeter exceeds a predetermined threshold.

2. The blood purification system according to claim 1, further comprising:
a pressure gauge configured to measure a pressure at a connection between the extracorporeal membrane oxygenation system and the blood drainage line or the blood return line, wherein
the controller further includes a circulation controller that reduces a flow rate of the flow rate adjuster to a predetermined flow rate in a case where a rate of change of the flow rate measured by the flowmeter exceeds the predetermined threshold and the pressure measured by the pressure gauge is within a predetermined range.

3. The blood purification system according to claim 2, further comprising:
a bypass line connecting a point upstream of the blood purification pump in the blood drainage line to the blood return line;
a first flow path switch disposed in a vicinity of a connection between the blood drainage line and the bypass line; and
a second flow path switch disposed in a vicinity of a connection between the blood return line and the bypass line, wherein
the controller includes a switch controller, and
in a case where a rate of change of the flow rate measured by the flowmeter exceeds the predetermined threshold and the pressure measured by the pressure gauge exceeds the predetermined range, the switch controller switches the second flow path switch so as to switch a flow path for liquid flowing through the blood return line to the bypass line, and switches the first flow path switch to stop an inflow of liquid from the extracorporeal membrane oxygenation system to the blood drainage line and to causes the liquid flowing through the bypass line to flow into the blood drainage line.

4. The blood purification system according to claim 1, further comprising:
a blood return pump provided to the blood return line, wherein
the upstream end of the blood drainage line is connected to a point downstream of the ECMO blood pump in the extracorporeal membrane oxygenation system, and
the downstream end of the blood return line is connected to a point downstream of the ECMO blood pump in the extracorporeal membrane oxygenation system.

5. The blood purification system according to claim 4, further comprising:
a pressure buffer provided at a point upstream of the blood return pump in the blood return line and capable of storing a predetermined amount of liquid; and
a buffer pressure gauge configured to measure a pressure of the pressure buffer, wherein
the controller further includes a flow rate controller that adjusts a flow rate of the flow rate adjuster and a flow rate of the blood return pump so as to control a value measured by the buffer pressure gauge to be within a predetermined range.

6. An intermediate system adapted to be provided at a connection between and extracorporeal membrane oxygenation system that includes an ECMO blood pump and an oxygenator disposed downstream of the ECMO blood pump, and a blood purification system that includes a blood purification pump, a blood purifier disposed downstream of the blood purification pump, a drain line for draining filtrate from the blood purifier, and a drain pump provided to the drain line,
the intermediate system comprising:
an intermediate blood drainage line having an upstream end that is connected to a point downstream of the ECMO blood pump in the extracorporeal membrane oxygenation system, and a downstream end that is connected to an upstream end of the blood purification system;
a flow rate adjuster provided to the intermediate blood drainage line and configured to adjust a flow rate in the intermediate blood drainage line;
an intermediate blood return line having an upstream end that is connected to a downstream end of the blood purification system, and a downstream end that is connected to a point downstream of the ECMO blood pump in the extracorporeal membrane oxygenation system;
a blood return pump provided to the intermediate blood return line;
a flowmeter configured to measure a flow rate in the extracorporeal membrane oxygenation system;
a notifier; and
a controller, wherein
in a case where a rate of change of the flow rate measured by the flowmeter exceeds a predetermined threshold, the controller determines that a problem has occurred in the extracorporeal membrane oxygenation system, and causes the notifier to notify the occurrence of the problem.

7. The intermediate system according to claim 6, further comprising:
a bypass line connecting a point upstream of the flow rate adjuster in the intermediate blood drainage line to the intermediate blood return line;
a first flow path switch disposed in a vicinity of a connection between the intermediate blood drainage line and the bypass line;
a second flow path switch disposed in a vicinity of a connection between the intermediate blood return line and the bypass line; and
a pressure gauge configured to measure a pressure at a connection between the extracorporeal membrane oxygenation system and the intermediate blood drainage line or the intermediate blood return line, wherein
the controller includes a switch controller, and
in a case where a rate of change of the flow rate measured by the flowmeter exceeds the predetermined threshold and the pressure measured by the pressure gauge exceeds a predetermined range, the switch controller switches the second flow path switch so as to switch a flow path for liquid flowing through the intermediate blood return line to the bypass line, and switches the first flow path switch to stop an inflow of liquid from the extracorporeal membrane oxygenation system to the intermediate blood drainage line and to causes the liquid flowing through the bypass line to flow into the intermediate blood drainage line.
